(19) United States
(12) Patent Application Publication
(10) Pub. No.: US 2024/0415880 A1
(43) Pub. Date: Dec. 19, 2024

(54) Treatment of Breast and Other Cancers Using Zinc Chloride Instead of Radiation

(71) Applicant: Norman A. Brooks, Encino, CA (US)

(72) Inventor: Norman A. Brooks, Encino, CA (US)

(21) Appl. No.: 18/603,593

(22) Filed: Mar. 13, 2024

Related U.S. Application Data
(60) Provisional application No. 63/472,947, filed on Jun. 14, 2023.

(51) Int. Cl. A61K 33/30; A61K 31/138; A61K 31/337; A61K 31/4745; A61K 31/513; A61K 33/243; A61K 36/66; A61K 38/14; A61K 38/21; A61K 47/38; A61M 25/00; A61M 25/10

(52) U.S. Cl. CPC A61K 33/30; A61K 31/138; A61K 31/337; A61K 31/4745; A61K 31/513; A61K 33/243; A61K 36/66; A61K 38/14; A61K 38/21; A61K 47/38; A61M 25/002; A61M 2025/105; A61M 2205/0238

(57) ABSTRACT

An improved method for treating breast cancer using zinc chloride as an alternative to radiation is provided. Zinc chloride is a deeply penetrating topical medicine that kills cancerous tissue equivalent to radiation. Radiation is time-consuming, costly, and can cause severe radiation-related adverse events, including secondary cancers, lung, and heart damage. An analysis of 20,000 women showed that radiation was able to reduce mortality by 13.2% from breast cancer, but this improvement was offset by radiation mortality from adverse events of 21.2% [Favorable and unfavorable effects on long-term survival of radiotherapy for early breast cancer: an overview of the randomized trials, Lancet 2,000:355: May 20, 2000]. This invention allows the surgeon to destroy residual malignant tissue that surrounds a breast cancer lumpectomy wound equivalent to radiotherapy without the mortality of radiation.

### BACKGROUND OF THE INVENTION

Zinc chloride is a locally applied anti-cancer medicine that deeply penetrates and kills tissue, used since 1835 suspended in inactive paste vehicles to treat breast and skin cancers. The paste vehicle has become known as black salve. The U.S. Food & Drug Administration recognizes zinc chloride not suspended in paste to be a generally safe substance, and has approved aqueous zinc chloride in low concentration for intravenous use. The treatment of an advanced melanoma excision wound and a breast cancer lumpectomy wound with aqueous zinc chloride protects against the potential spread of cancer at the time of surgery. Surgery opens blood vessels and reduces cancer immunity. Case reports of 103 consecutive advanced melanomas treated with zinc chloride combined with surgery show a significant and statistical (p=0.003) improvement in cure rates over conventional surgery of melanomas. Cases are presented showing remarkable healing of locally advanced breast cancer tumors treated with zinc chloride. In breast cancer lumpectomy only a narrow rim of tissue is removed around the tumor with the inability to remove all the malignant tissue. Radiation has become the standard of treatment, and is used to kill residual malignant tissue that remains in the patient. Further research of a weak concentration of aqueous zinc chloride, the active ingredient of the black salve, is indicated as an alternative to radiation in breast cancer surgery. Radiation in breast cancer lumpectomy is time consuming, costly, and can cause severe radiotherapy related adverse events, including rare but serious second cancers, lung, and heart damage.

The preceding paragraph is the abstract from the inventor's published journal article titled, Protection against Spread of Cancer in Breast Cancer and Melanoma using Aqueous Zinc Chloride to Kill Residual Malignant Tissue at the Time of Surgery (Brooks, Ann Case Rep: 8: 1264, 17 Apr. 2023) which is hereby incorporated by this reference.

### SUMMARY OF INVENTION

Zinc chloride has been used for almost 200 years to treat breast cancers, but the vast majority of modern medical practitioners are unaware of the proper dosage required for treatment. As a result, zinc chloride is rarely used today. Accordingly, what is needed is an improved method of applying, administering, storing and dosing zinc chloride for the treatment of breast cancer following a lumpectomy or other similar breast conserving surgery.

Zinc chloride readily dissolves in distilled or purified water to form an aqueous solution of zinc chloride, the active ingredient of this invention. The concentration can vary from less than 0.1% to greater than 50%. Zinc chloride is available as zinc chloride granular USP which is a manufactured product. Zinc chloride is an extremely potent and deeply penetrating topical agent that destroys cancerous tissue. Application in proper dosage which can vary in different areas of a breast cancer lumpectomy wound is the essence of this invention. The bottom of a lumpectomy wound overlies the pleural lining of the lungs and chest cavity. Care is taken to avoid the possible but very unlikely event of zinc chloride penetration into the pleural cavity.

After a breast cancer is definitively diagnosed by biopsy, the tumor is often treated by lumpectomy or other breast conserving surgery. Radiation is given either at the time of surgery, or delayed, and is used to destroy residual malignant cells that exist surrounding the narrow margins of a lumpectomy or breast conserving surgery. Treatment with radiation at the time of surgery is advantageous over the much more commonly used delayed radiation because malignant tumor cells are treated before they have a chance to proliferate. Zinc chloride offers the advantage of destroying cancerous cells at the time of surgery, and is much simpler to administer than the cumbersome intraoperative radiation, and has no radiation hazards or systemic toxicity. (See Discussion—Zinc Chloride vs. Radiation in Breast Cancer Lumpectomy—Annals of Case Reports—Background of Invention).

Zinc chloride may be applied manually to a lumpectomy wound or a balloon applicator may be utilized (to be discussed). Pre-packaging of zinc chloride in containers specifying dosage is essential for the correct amount of zinc chloride to be applied to a lumpectomy wound. The depth of penetration of zinc chloride depends on the amount and concentration of zinc chloride applied.

Historically, zinc chloride has been used suspended in paste vehicles. Mohs, the surgeon referred to in the Annals of Case Reports, used a paste consisting of 34.5 ml zinc chloride saturated solution, 40 g of stibnite, and 10 g of Sanguinaria Canadensis (Bloodroot plant). Sanguinaria is available in a USP powder containing the anti-cancer alkaloid sanguinarine. Tsukada, also referred to in the Annals of Case Reports, substituted the inert fillers of zinc oxide starch powder and glycerin for the sanguinaria and stibnite of Mohs paste, to make the paste that effectively healed the massive breast cancer wound shown in the photographs in the Results section of the Annals Report. Despite the massive size of the cancerous wound, no pleural penetration of zinc chloride was reported. Cellulose, a plant based polysaccharide closely related to starch, has also been used to make an effective paste. Tagachi et al, studied a modified Mohs paste using cellulose and compared it to the original Mohs paste, and found in mice using computed tomography scans that the cellulose modified paste allowed for a deeper penetration of zinc chloride than the Mohs paste. Mohs reported a depth of zinc chloride paste penetration ranging from a fraction of a millimeter to 3.0 centimeter depending on the thickness and diameter of the paste application (Mohs F E, Chemosurgery. Charles C Thomas 1978;p. 12). Badshah reported a bacterial derived cellulose matrix that is non-toxic, biodegradable, simple to produce and purify, and forms into a nanofibrillar moldable network gum structure in which zinc chloride can be suspended. (Badshah et al. Surface modification and evaluation of bacterial cellulose for drug delivery. International Journal of Biological Macromolecules July 2018 pages 526-533). Zinc chloride suspended in this moldable cellulose gum is particularly advantageous for the topical treatment of breast cancer lumpectomy or other conservative breast cancer excision wounds. Alternative paste formulas have substituted different fillers, thickeners and binders such as titanium dioxide, turmeric, polyethylene glycol, and galangal but none have been as efficacious as cellulose.

The inventor has explored enhancing Mohs paste with the anti-cancer alkaloids of Paclitaxel (Taxol) derived from the Yew tree (Taxus brevifelix, Taxus yannanensis). In a previous now expired patent dated 24 Aug. 2000 #WO 00/48541 entitled Zinc Chloride in Treating Skin Diseases, the inventor found the following:
“Paclitaxel zinc chloride paste concentrations of 0.01%, 0.1%, 1%, and 10% have been experimentally applied to pig skin in varying thicknesses from paper thin to 1 mm thick over surface areas varying from 4 mm to 1.5 cm in diameter to determine whether the addition of the paclitaxel affects the zinc chloride penetration. No difference in depth of penetration was observed for any of the concentrations, different surface areas, or thicknesses when compared to zinc chloride fixative paste without paclitaxel. The preservative property of zinc chloride is believed to maintain the activity of the plant alkaloids indefinitely.”

It is noted that the same advantages of zinc chloride over radiation hold for other cancers in which radiation has been used including but not limited to bladder cancers, uterine cancers, cancers of the vaginal cavity and cervix, rectal cancers, esophageal cancers, prostate cancers, intra-cavitary melanomas, melanomas, pancreatic cancers, and head and neck cancers. Mohs successfully treated cancers of the mouth, cancers of the accessory nasal sinuses, cancer of the larynx, cancer of the parotid glands and other salivary glands, cancer of the anal region, cancer of the vulva and accessible sarcomas with zinc chloride. {Mohs F E. Chemosurgery Charles C. Thomas: Springfield pg. 179-223)

### BRIEF DESCRIPTION OF THE DRAWINGS

FIG. 1. Unit dose packaging

FIG. 2. Unit dose packaging

FIG. 3. Unit dose packaging

FIG. 4. Zinc chloride gum mold packaging

FIG. 5. Zinc chloride gum mold packaging

FIG. 6. Zinc chloride packaged gum mold

FIG. 7. Medicated balloon applicator

FIG. 8. Steps in lumpectomy treatment with zinc chloride

### DETAILED DESCRIPTION OF THE INVENTION

### Embodiments

5 embodiments are described. Research and clinical trials are needed to establish these embodiments as alternatives to radiation for breast cancer.

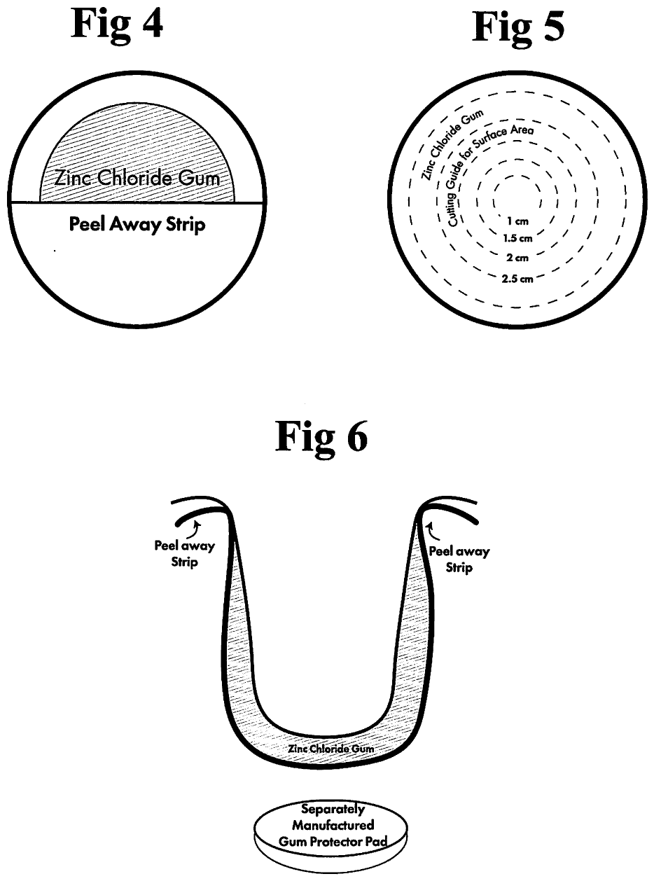

1. Tsukada paste. The Tsukada paste consists of 10 g zinc chloride in 10 ml purified water mixed with 5 g zinc oxide starch powder and 5-10 ml glycerin. This results in a 40%-50% concentration of zinc chloride by weight which is too strong for use on a lumpectomy wound. Only 4 g of zinc chloride should be mixed into this formulation. The Tsukada paste is simple to manufacture and easy to manually apply and does not contain any biologically active ingredients except zinc chloride. The Tsukada paste was used successfully to heal the large ulcerated breast cancer wound shown in FIGS. 4 and 5 of the Results Section of the journal article incorporated by reference in the Background of the Invention. As previously stated, care is taken to avoid dispersion of zinc chloride into the pleural cavity, but no damage to the pleural cavity was reported in this case despite the massive size of the cancerous wound and the high 40-50% concentration of zinc chloride applied.
2. Mohs paste. Mohs used a paste consisting of 34.5 ml zinc chloride saturated solution, 40 g of stibnite, and 10 g of Sanguinaria Canadensis (Bloodroot plant) with a zinc chloride concentration of 45% of the preparation by weight. {Mohs F E. Chemosurgery Charles C. Thomas: Springfield pg. 5) This concentration is too strong for use on a lumpectomy wound. Stibnite is a derivative of antimony and cannot be formulated to USP standards. The inventor modified the Mohs paste using 34.5 ml of 40% zinc chloride aqueous solution and eliminated the stibnite using only 40 g of Sanguinaria (Bloodroot Plant) instead. The zinc chloride mixed well into the powdered Sanguinaria USP resulting in a flexible and fully acceptable paste for topical application. The Mohs formula results in a large paste mixture, the quantity is much greater than that needed for individual patient use. A problem with Mohs paste is that Mohs kept it in a large jar with enough paste to treat many patients.

There was no packaging or dose specificity. Furthermore, the Mohs paste jar was not sealed and the paste could become too dry and hard or become too watery and runny depending on the atmospheric conditions. Sealed unit dose packaging is needed.

3. Manually Applied Aqueous Zinc Chloride Solution or Paste. In this embodiment aqueous zinc chloride solution or aqueous zinc chloride suspended in a paste or other mixture is manually applied to a lumpectomy wound. The solution or paste suspension is obtained in dose specific amounts from sealed pre-packaged containers. The dose and location to be applied is designated on each container. The paste can be applied with sterile gauze or gloved hand, the aqueous solution is applied with sterile gauze.

The technique in this embodiment (3) may be a 2-step procedure. First, amylopectin (e.g. BleedStop™, OTC FDA cleared), a potent hemostatic plant polysaccharide closely related to starch and cellulose, may be manually applied to the wound to achieve hemostasis. Effective hemostasis can often be achieved in seconds to less than a minute. Large vessels can be sutured closed with absorbable suture. After hemostasis has been obtained the aqueous zinc chloride solution or aqueous zinc chloride suspended in a paste or other mixture is manually applied in dose specific amounts to different areas of the lumpectomy wound. Mild manual compression can be used to disperse the medicinal zinc chloride into the cancerous tissues. Alternatively, a non-medicated expandable balloon compressor with a cannula to fill the balloon at the time of surgery can be inserted against the applied medicinal zinc chloride and filled with air or saline to create pressure compression to disperse the zinc chloride into the tissues.

4. Manual Application of Molded Zinc Chloride Gum. In this embodiment aqueous zinc chloride is suspended at least in part of a cellulose gum. (Badshah et al. Surface modification and evaluation of bacterial cellulose for drug delivery. International Journal of Biological Macromolecules July 2018 pages 526-533). The bacterial cellulose in the Badshah report is non-toxic, biodegradable, simple to produce and purify, forms into a nanofibrillar moldable network gum structure in which zinc chloride can be suspended and is an effective drug delivery system. Taguchi reported that a modified Mohs paste containing cellulose has a greater depth of penetration compared to the original Mohs paste. The zinc chloride gum is moldable and can be made in various sizes and shapes for manual placement in a lumpectomy wound. The gum is biodegradable and absorbable by the body.
5. Balloon Application of Molded Zinc Chloride Gum. A medicated zinc chloride balloon applicator replaces manual application. A balloon applicator consists of a synthetic expandable impermeable balloon that is adhesive on the outer surface. On this outer surface a permeable zinc chloride gum mold or other mixture is adhered to the balloon in a thin layer. The balloon is filled with saline or air at the time of surgery through a cannula to conform the balloon to the cavity of the lumpectomy wound, and to create compression. The compression creates pressure that disperses the medicinal zinc chloride into the cancerous tissues. A balloon applicator is manufactured in different sizes and shapes to fit lumpectomy wounds.

Although embodiments have been described in detail, various modifications may be made without departing from the scope and spirit of the invention. For example, a mixture of agents derived from naturally occurring minerals and plants have been described, (zinc chloride, Sanguinaria canadensis (Bloodroot plant), Taxol (Paclitaxel) from the Yew tree, bacterial cellulose, and amylopectin derived from plants), but similar agents can be synthesized (ChemPilots.com). Pharmacologically active compounds can be added to the zinc chloride mixture, or zinc chloride can be modified to a zinc hydrochloride variant. The drawings are presented as examples and variations of these drawings can be made. Accordingly, the invention is not to be limited, except as by the general concepts presented for effective dose specific application of medicinal zinc chloride to a lumpectomy wound as an alternative to radiation.

### EXPLANATION OF DRAWINGS

Pre-packaged sealed dose specific containers and applicators are illustrated in the following drawings. The designated dosage in the individual containers illustrated are for conceptual purposes and require research to determine the exact optimal dosage.

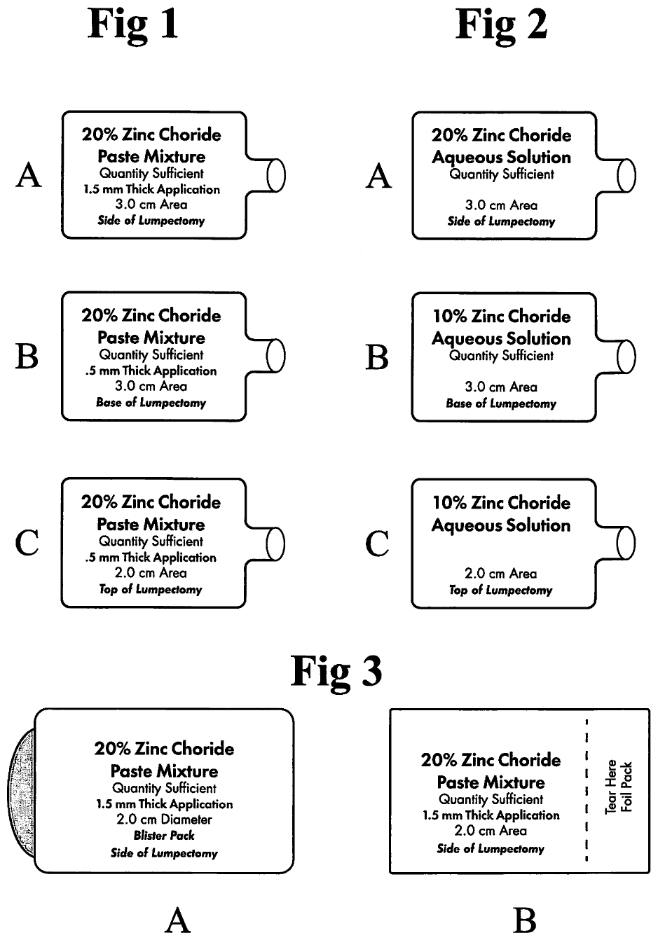

FIGS. 1A-C and 2A-C illustrate unit dose packaging of aqueous zinc chloride solution and mixtures of aqueous zinc chloride (mixtures refer to paste or matrix formulations of zinc chloride). The packages are sealed. The individual containers specify the dosage, location, and area size of the lumpectomy wound the zinc chloride is to be applied. The paste is applied with gloved hand or applied with sterile 2×2 gauze. Aqueous zinc chloride is applied with gauze. Mild manual compression is used to disperse the medicinal zinc chloride into the cancerous tissues.

FIGS. 3A & 3B illustrate alternative pre-packaged Blister-Pack and foil pack containers. The containers designate the dosage, location, and area size of the lumpectomy wound the zinc chloride is to be applied.

FIG. 4 illustrates a zinc chloride gum that can be removed from the container, molded, and be directly applied to the lumpectomy wound.

FIG. 5 shows a zinc chloride gum that can be cut open with a scissors. The concentric rings serve as a cutting guide for precise surface area application.

FIG. 6 illustrates a zinc chloride gum mold that can fit into and fill a lumpectomy wound for drug delivery. The gum mold can be manufactured in different sizes and shapes. The gum is manually inserted into the wound with mild compression to disperse the zinc chloride into the cancerous tissues. A non-medicated balloon compressor with a cannula for inflation or expansion at the time of surgery can also be used to disperse the zinc chloride. A separately manufactured non-medicated semi-permeable cellulose gum pleural protector pad is shown. The pad may be placed at the bottom of the lumpectomy wound to limit the dispersion of zinc chloride into the tissues above the pleural cavity prior to inserting the zinc chloride gum mold. The illustration shows the applicator packaged in sealed peel-away strips.

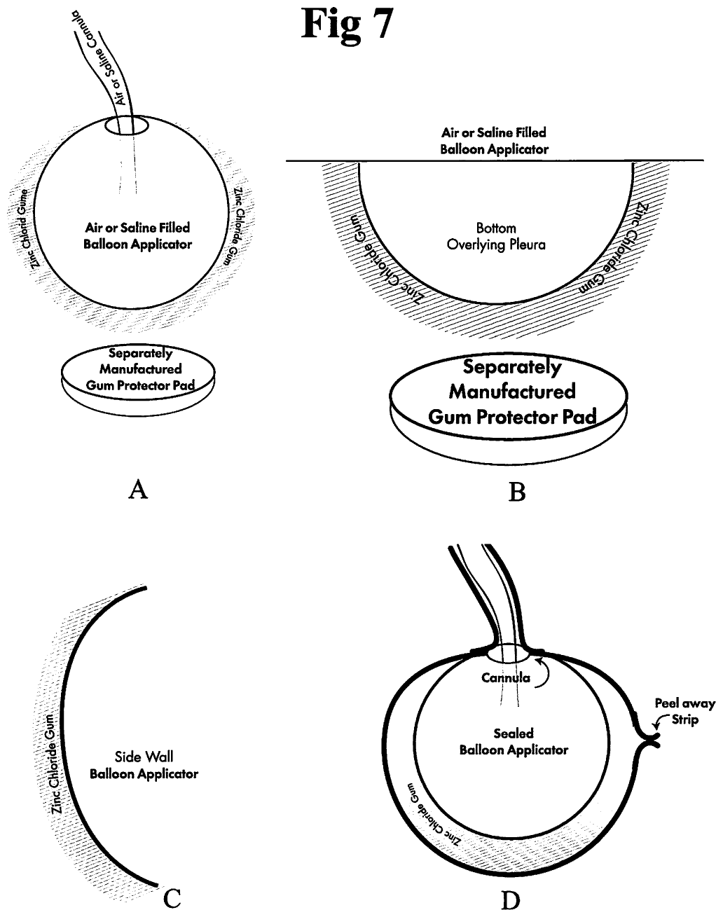

FIG. 7 shows a medicated zinc chloride gum balloon applicator that replaces manual application. The applicator consists of an impermeable balloon with a permeable zinc chloride cellulose gum adhered to the outer adhesive surface of the balloon. The balloon is filled with saline or air through a cannula at the time of surgery to conform the balloon to the lumpectomy wound and create pressure. The pressure disperses the zinc chloride into the cancerous tissues. Also shown is a separately manufactured non-medicated semi-permeable gum protector pad that is placed at the bottom of the lumpectomy wound prior to use of the zinc chloride balloon to limit zinc chloride dispersion into the tissues overlying the pleural cavity. The balloon is shaped so as to taper off the zinc chloride gum to zero at the top of the balloon at the cannula to protect the overlying skin from zinc chloride dispersion. Bottom views, side views, separately manufactured gum protector pad and packaging and sealing of the inflatable or expandable zinc chloride gum balloon are shown.

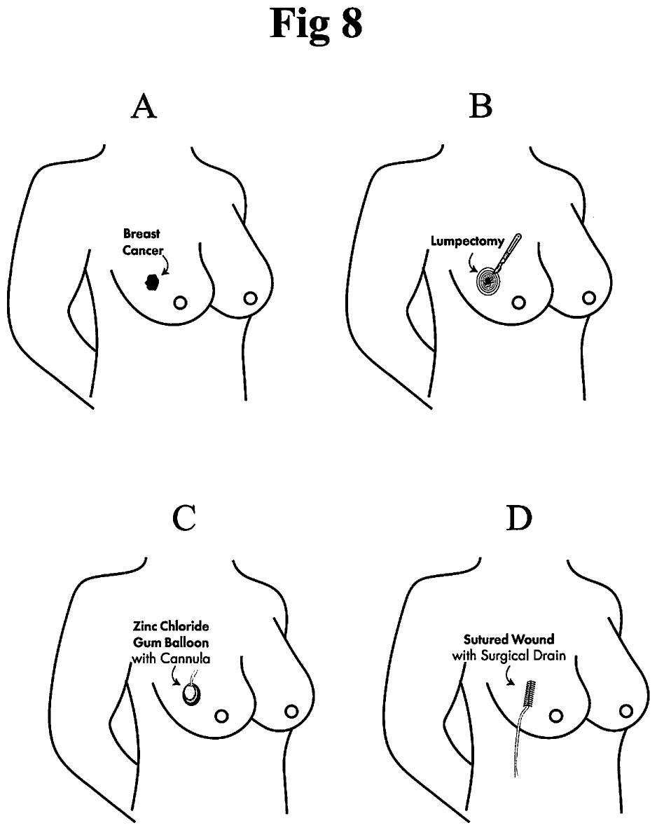

FIGS. 8A-B illustrate a lumpectomy excision of a breast cancer. FIG. 8C shows the insertion of an inflatable or expandable balloon applicator compressing a medicinal zinc chloride mixture or gum mold into the cancerous tissues. FIG. 8D illustrates suture closure of the wound with a surgical drain inserted.

1-16. (canceled)

17. An article of manufacture, comprising:
a package designed to store a unit dose of a medication;
a composition comprising zinc chloride or zinc hydrochloride dissolved as an aqueous solution or suspended in a pharmaceutical carrier and sealed within the package;
wherein the concentration of zinc chloride or zinc hydrochloride in the composition is between 0.1% and 50%.

18. The article of manufacture of claim 17, wherein the package comprises written instructions for applying the composition to a wound of a lumpectomy or other breast cancer conserving surgery as to the location and area of the wound, with a lower dosage to be applied at a bottom portion of the wound overlying the pleura and at a top portion of the wound underlying the skin.

19. The article of manufacture of claim 17, wherein the pharmaceutical carrier is a paste, gum or matrix mixture.

20. The article of manufacture of claim 19, wherein the composition further comprises one or more inert paste or matrix fillers, thickeners, and binders chosen from polysaccharide polymers of cellulose and zinc oxide starch powder, closely related hemostatic polysaccharide polymer amylopectin, titanium dioxide, turmeric, polyethylene glycol, galangal, and glycerin, or equivalent synthetic fillers and binders.

21. The article of manufacture of claim 19, wherein the composition further comprises one or more immunologically or pharmacologically active additives chosen as examples but not limited to powdered Sanguinaria canadensis USP containing sanguinarine and other anti-cancer alkaloids, anti-cancer alkaloids of Paclitaxel, fluorouracil, imiquimod, bleomycin, interferon, tamoxifen, and cisplatin.

22. The article of manufacture of claim 17, wherein the pharmaceutical carrier is a solvent or a paste, gum or matrix mixture and the zinc chloride or zinc hydrochloride is present in the composition at a weight percentage of 20%.

23. The article of manufacture of claim 17, wherein the pharmaceutical carrier is a moldable gum comprising at least in part of cellulose or a synthetic matrix capable of being molded and manually placed or inserted into a lumpectomy wound in different sizes and shapes, and capable of being compressed to disperse the zinc chloride or zinc hydrochloride into cancerous tissues surrounding the lumpectomy wound.

24. The article of manufacture of claim 17, wherein the carrier comprises at least in part of a bacterial derived cellulose gum.

25. A kit comprising the article of manufacture of claim 19 and a device comprising a balloon compressor designed to compress the zinc chloride or zinc hydrochloride in the paste, gum or matrix mixture into cancerous tissues surrounding a lumpectomy wound, the balloon compressor comprising a synthetic impermeable non-medicated expandable membrane and a cannula configured to fill the balloon with a liquid or gas.

26. The kit of claim 25, wherein the pharmaceutical carrier is a moldable gum and further comprises amylopectin or other hemostatic agent.

27. A device comprising:
a balloon compressor comprising:
an inflatable or expandable impermeable balloon, wherein the balloon is coated with a zinc chloride or zinc hydrochloride mixture and is designed to disperse the zinc chloride or zinc hydrochloride into cancerous tissues surrounding a lumpectomy wound by compression pressure; and
a cannula configured to fill and expand the balloon with a liquid or gas.

28. The device of claim 27, wherein the zinc chloride or zinc hydrochloride mixture comprises a pharmaceutical carrier comprising at least in part of a cellulose gum.

29. A device comprising:
a balloon applicator comprising:
an inflatable or expandable impermeable balloon coated with a composition comprising zinc chloride or zinc hydrochloride suspended in a gum, paste, or matrix mixture;
a cannula configured to fill and expand the balloon with a liquid or gas;
a non-medicated semi-permeable cellulose gum pleural protector pad, matrix, or synthetic pad designed to be placed at the bottom of a lumpectomy wound prior to insertion of the balloon applicator to limit dispersion of the zinc chloride or zinc hydrochloride into tissues overlying the pleura,
wherein the balloon applicator is designed to taper the zinc chloride or zinc hydrochloride gum, paste, or matrix at a top portion of the balloon to limit dispersion of zinc chloride or zinc hydrochloride into overlying skin.

30. A method of treating a breast cancer lumpectomy wound or other conservative breast cancer excision wound, the method comprising:
limiting or stopping bleeding of the wound by applying hemostatic amylopectin powder to the wound, an absorbable suture for large blood vessels, and/or a non-medicated balloon compressor;.
applying a dose of zinc chloride or zinc hydrochloride obtained from an article of manufacture of claim 17 manually with a gloved hand or sterile gauze, or by way of a kit of claim 25, or by way of a device of claim 27;
optionally, applying a non-medicated semi-permeable cellulose gum protector pad, matrix, or synthetic pad at the bottom of the lumpectomy wound to limit dispersion of zinc chloride or zinc hydrochloride into tissues above the pleural cavity.

31. A method comprising:
performing a lumpectomy or other conservative breast conserving surgery to remove a breast cancer tumor;
limiting or stopping bleeding of a wound from the lumpectomy or other surgery by applying hemostatic amylopectin powder to the wound, an absorbable suture for large blood vessels, and/or a non-medicated balloon compressor;
applying a non-medicated semi-permeable cellulose gum protector pad, matrix, or synthetic pad at the bottom of the lumpectomy wound; and
applying zinc chloride or zinc hydrochloride to the wound as a composition having a solution, paste or gum as a pharmaceutical carrier by manual compression or by way of a balloon compressor, or applying zinc chloride or zinc hydrochloride to the wound by way of a gum, paste, or matrix medicated balloon applicator.

32. A method, comprising:
applying a dose-specific zinc chloride or zinc hydrochloride mixture or gum balloon zinc chloride or zinc hydrochloride medicated applicator for assisted administration, a dose-specific zinc chloride or zinc hydrochloride moldable gum or mixture for manual application, or a zinc chloride or zinc hydrochloride dose-specific aqueous solution or mixture in packaged and sealed containers to a tumor or post-operative surgical site from which a tumor has been removed;
wherein the dose of zinc chloride or zinc hydrochloride is obtained by way of an article of manufacture of claim 17, applied and compressed manually, or with a kit of claim 25, a device of claim 27, or a device of claim 29;
wherein the tumor is chosen from a bladder tumor, a uterine tumor, a vaginal tumor, a cervix tumor, an anal or rectal tumor, an esophageal tumor, a prostate tumor, a melanoma tumor, a pancreatic tumor, and a head and neck tumor including a tumor of the mouth, the accessory nasal sinuses, the larynx, the parotid glands, and other salivary glands.
